(19) United States
(12) Patent Application Publication
(10) Pub. No.: US 2026/0053626 A1
(43) Pub. Date: Feb. 26, 2026

(54) DERMAL CAP FOR RESURFACING BONE JOINTS

(71) Applicant: TheraMicro, Memphis, TN (US)

(72) Inventors: Alan G. Taylor, Memphis, TN (US); Rebecca Hawkins Wahl, Escondido, CA (US)

(73) Assignee: TheraMicro, Memphis, TN (US)

(21) Appl. No.: 19/310,733

(22) Filed: Aug. 26, 2025

Related U.S. Application Data
(60) Provisional application No. 63/687,251, filed on Aug. 26, 2024.

(51) Int. Cl. A61F 2/30; A61F 2/42

(52) U.S. Cl. CPC A61F 2/30734; A61F 2/30749; A61F 2/30771; A61F 2/3094; A61F 2/4225; A61F 2/4241; A61F 2002/30736; A61F 2002/30772; A61F 2002/30884; A61F 2002/3093

(57) ABSTRACT

An apparatus and methods are provided for a dermal cap for resurfacing a metatarsal head of the foot or a metacarpal head of the hand. The dermal cap comprises an outer surface having a convex shape, an inner surface having a concave shape, and a rounded peripheral surface joining the inner surface and the outer surface. The inner surface can be placed onto a bone head such that the outer surface provides a bone head surface resembling the original articular cartilage. The outer surface effectively restores the articular cartilage and thus relieves pain and facilitates better joint function. The dermal cap can be formed by stretching it over a rounded mandrel shaped similarly to the size and anatomy of the bone head to be treated. The dermal cap can be freeze-dried on the rounded mandrel to set the desired anatomy of the dermal cap.

### PRIORITY

This application claims the benefit of and priority to U.S. Provisional Application, entitled “Dermal Cap For Resurfacing Bone Joints,” filed on Aug. 26, 2024, and having application Ser. No. 63/687,251, the entirety of said application being incorporated herein by reference.

### FIELD

Embodiments of the present disclosure generally relate to medical implants. More specifically, embodiments of the disclosure relate to an apparatus and methods for a dermal cap for resurfacing a metatarsal head of the foot or metacarpal head of the hand.

### BACKGROUND

Articular cartilage is a smooth, white tissue which covers the ends of bones where they come together to form joints in humans and many animals so as to facilitate articulation of the joints and protect and cushion the bones. Cartilage may become damaged, however, due to abrupt trauma or prolonged wear. A number of surgical techniques have been developed to treat damaged cartilage. Restoring articular cartilage is known to relieve pain and facilitate better joint function, as well as potentially delaying or preventing an onset of arthritis. As such, there is an ongoing need for the development of osteochondral treatment capabilities such as those found in, for example, treating damage to articular cartilage in joints. Provided herein are embodiments and methods for a dermal cap for treating osteochondral defects by resurfacing a metatarsal head of the foot or a metacarpal head of the hand.

### SUMMARY

An apparatus and methods are provided for a dermal cap for resurfacing a metatarsal head of the foot or metacarpal head of the hand. The dermal cap comprises an outer surface having a convex shape, an inner surface having a concave shape, and a rounded peripheral surface joining the inner surface and the outer surface. The inner surface is configured to be placed into contact with a metatarsal head or a metacarpal head such that the outer surface provides a bone head surface resembling the original articular cartilage. The outer surface is configured to effectively restore the articular cartilage and thus serves to relieve pain and facilitate better joint function, as well as potentially delay or prevent an onset of arthritis. The dermal cap is configured to be formed by stretching it over a rounded mandrel that is shaped similarly to the size and anatomy of a bone head to be treated. The dermal cap can be freeze-dried on the rounded mandrel to set the desired anatomy of the dermal cap.

In an exemplary embodiment, an apparatus for a dermal cap for treating osteochondral defects of metatarsal bones of the feet or metacarpal bones of the hands comprises: an outer surface having a convex shape; an inner surface having a concave shape; and a rounded peripheral surface joining the inner surface and the outer surface.

In another exemplary embodiment, the inner surface is configured to be placed into contact with a metatarsal head or a metacarpal head such that the outer surface provides a bone head surface resembling the original articular cartilage. In another exemplary embodiment, the outer surface is configured to effectively restore the articular cartilage and thus serves to relieve pain and facilitate better joint function, as well as potentially delay or prevent an onset of arthritis.

In another exemplary embodiment, the dermal cap is configured to be formed by stretching it over a rounded mandrel that is shaped similarly to the size and anatomy of a bone head to be treated. In another exemplary embodiment, the dermal cap is configured to be freeze-dried on the rounded mandrel to set the desired anatomy of the dermal cap.

In another exemplary embodiment, the dermal cap further includes pre-cut holes disposed around a circumference of the dermal cap adjacent to the rounded periphery surface. In another exemplary embodiment, the pre-cut holes are configured to facilitate using soft tissue anchors or sutures to retain the position and configuration of the dermal cap on the bone head being treated. In another exemplary embodiment, the dermal cap further includes a tab and a pre-cut hole that are configured to facilitate using soft tissue anchors or sutures to mount the dermal cap onto the bone head being treated.

In another exemplary embodiment, the dermal cap is configured to be implemented in various sizes to accommodate the anatomy of the specific bone joints to be treated. In another exemplary embodiment, the dermal cap has a diameter that ranges between about 10 mm and about 24 mm. In another exemplary embodiment, the dermal cap is non-perforated. In another exemplary embodiment, the dermal cap is perforated to aid bone tissue with fusing to the material comprising the dermal cap. In another exemplary embodiment, the material comprising the dermal cap comprise any synthetic or natural homogenous material suitable for implantation into bone. In another exemplary embodiment, the material comprises human or animal allograft.

In an exemplary embodiment, a method for a dermal cap for treating osteochondral defects of metatarsal bones of the feet or metacarpal bones of the hands comprises: forming an outer surface having a convex shape; forming an inner surface having a concave shape; and joining the inner surface and the outer surface by way of a rounded peripheral surface.

In another exemplary embodiment, forming the inner surface includes forming the inner surface to be placed into contact with a metatarsal head or a metacarpal head such that the outer surface provides a bone head surface resembling the original articular cartilage. In another exemplary embodiment, forming the outer surface includes configuring the outer surface to restore the articular cartilage and thus to relieve pain and facilitate better joint function, as well as potentially delay or prevent an onset of arthritis.

In another exemplary embodiment, the method further includes disposing pre-cut holes around a circumference of the dermal cap adjacent to the rounded periphery surface. In another exemplary embodiment, disposing the pre-cut holes includes configuring the pre-cut holes to facilitate using soft tissue anchors or sutures to retain the position and configuration of the dermal cap on the bone head being treated. In another exemplary embodiment, the method further includes configuring a tab and a pre-cut hole to facilitate using soft tissue anchors or sutures to mount the dermal cap onto the bone head being treated.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates a first exemplary embodiment of a dermal cap for resurfacing a metatarsal head of the foot or metacarpal head of the hand, according to the present disclosure;

FIG. 2 illustrates a second exemplary embodiment of a dermal cap for resurfacing a metatarsal head of the foot or metacarpal head of the hand in accordance with the present disclosure;

FIG. 3 illustrates a side view of a first metatarsal bone that includes a head that may be resurfaced by way of a dermal cap, according to the present disclosure; and

FIG. 4 illustrates a side view of a first metacarpal bone that includes a head that may be resurfaced by way of a dermal cap, according to the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the dermal cap and methods disclosed herein may be practiced without these specific details. In other instances, specific numeric references such as “first surface,” may be made. However, the specific numeric reference should not be interpreted as a literal sequential order but rather interpreted that the “first surface” is different than a “second surface.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Articular cartilage is a smooth, white tissue which covers the ends of bones where they come together to form joints in humans and many animals so as to facilitate articulation of the joints and protect and cushion the bones. Cartilage may become damaged, however, due to abrupt trauma or prolonged wear. Restoring articular cartilage relieves pain and facilitates better joint function, as well as potentially delaying or preventing an onset of arthritis. As such, there is an ongoing need for the development of osteochondral treatment capabilities for treating damaged articular cartilage in joints. Provided herein are embodiments and methods for a dermal cap for treating osteochondral defects by resurfacing a metatarsal head of the foot or metacarpal head of the hand.

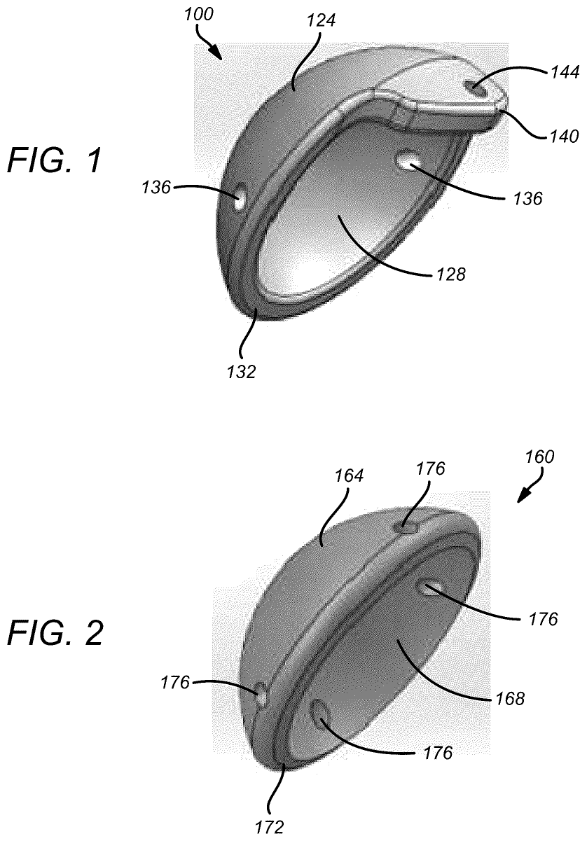

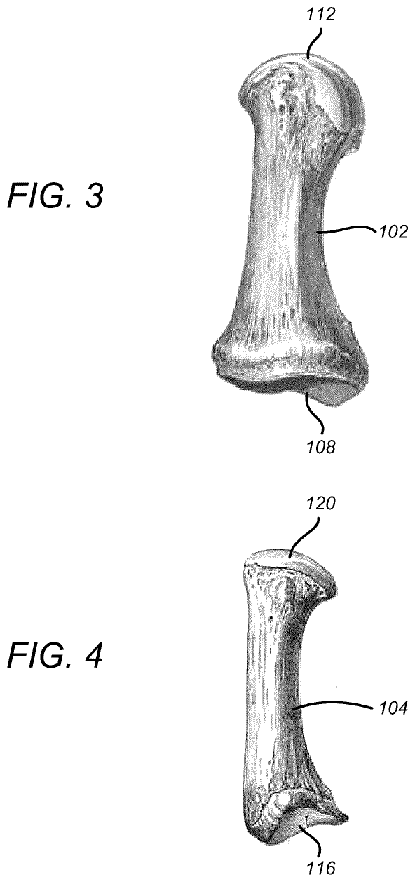

FIG. 1 illustrates an exemplary embodiment of a dermal cap 100 that is adapted for treating osteochondral defects of metatarsal bones of the feet or metacarpal bones of the hands. An exemplary 1^{st} metatarsal bone 102 that may be treated by way of the dermal cap 100 is shown in FIG. 3. An exemplary 1^{st} metacarpal bone 104 that may be treated by way of the dermal cap 100 is shown in FIG. 4. The 1^{st} metatarsal bone 102 has a base 108 and a metatarsal head 112. The base 108 articulates primarily with the medial cuneiform while the metatarsal head 112 articulates with the sesamoid bones. Similarly, the 1^{st} metacarpal bone 104 includes a base 116 and a metacarpal head 120. The base 116 articulates with the carpal bones and adjoining metacarpal bones while the metacarpal head 120 articulates with the proximal phalanx. In particular, the metatarsal head 112 and the metacarpal head 120 are covered with articular cartilage that may be damaged due to abrupt trauma or prolonged wear.

The dermal cap 100 shown in FIG. 1 is advantageously configured to resurface a damaged metatarsal head 112 or a metacarpal head 120 to restore joint function. As shown in FIG. 1, the dermal cap 110 includes an outer surface 124 and an inner surface 128 that are joined by a rounded peripheral surface 132. The outer surface 124 is convex while the inner surface 128 is concave, giving the dermal cap 100 its cap-shape. Thus, the inner surface 128 can be placed into contact with the metatarsal head 112 or the metacarpal head 120 such that the outer surface 124 provides a bone head surface resembling the original articular cartilage. As will be appreciated, resurfacing the bone head by way of the outer surface 124 effectively restores the articular cartilage and thus serves to relieve pain and facilitate better joint function, as well as potentially delay or prevent an onset of arthritis.

In some embodiments, the dermal cap 100 can be formed by stretching it over a rounded mandrel that is shaped similarly to the size and anatomy of the bone head to be treated. Once the desired anatomy has been achieved, the dermal cap 100 can be freeze-dried on the rounded mandrel to set the anatomy of the dermal cap 100. As shown in FIG. 1, the dermal cap 100 includes pre-cut holes 136 disposed around a circumference of the dermal cap 100 adjacent to the rounded periphery surface 132. The pre-cut holes 136 are configured to facilitate using soft tissue anchors or sutures to retain the position and configuration of the dermal cap 100 on the bone head being treated. Further, the dermal cap 100 includes a tab 140 and a pre-cut hole 144 that are configured to facilitate using soft tissue anchors or sutures to mount the dermal cap 100 onto the bone head, as described herein.

As mentioned herein, the dermal cap 100 can be adapted for treating the metatarsal head 112, shown in FIG. 3, or the metacarpal head 120, shown in FIG. 4, to restore joint function. As such, the dermal cap 100 may be implemented in various sizes to accommodate the anatomy of the specific bone joints to be treated. In some embodiments, the dermal cap 100 has a diameter that ranges between about 10 mm and about 24 mm. Further, the dermal cap 100 may be non-perforated as shown in FIG. 1, or in some embodiments, the dermal cap 100 may be perforated to aid bone tissue with fusing to the dermal cap 100. Further, the dermal cap 100 may comprise any synthetic or natural homogenous material suitable for implantation into bone, including human or animal allograft, and the like.

FIG. 2 illustrates an exemplary embodiment of a dermal cap 160 that is adapted for treating osteochondral defects of metatarsal bones of the feet or metacarpal bones of the hands. The dermal cap 160 shown in FIG. 2 is substantially similar to the dermal cap 100, shown in FIG. 1, with the exception that the dermal cap 160 lacks the tab 140 and the pre-cut hole 144. As shown in FIG. 2, the dermal cap 160 is adapted to resurface a damaged bone head, such as the metatarsal head 112, shown in FIG. 3, or the metacarpal head 120, shown in FIG. 4. The dermal cap 160 includes an outer surface 164 and an inner surface 168 that are joined by a rounded peripheral surface 172. The outer surface 164 is convex while the inner surface 168 is concave, giving the dermal cap 160 a cap-like shape. As such, the inner surface 168 can be placed into contact with the metatarsal head 112 or the metacarpal head 120 such that the outer surface 164 provides a bone head surface that effectively restores the articular cartilage and thus serves to relieve pain and facilitate better joint function, as well as potentially delay or prevent an onset of arthritis.

In some embodiments, the dermal cap 160 can be formed by stretching it over a rounded mandrel that is shaped similarly to the size and anatomy of the bone head to be treated. Once the desired anatomy has been achieved, the dermal cap 160 can be freeze-dried on the rounded mandrel to set the target anatomy of the bone head. As shown in FIG. 2, the dermal cap 160 includes pre-cut holes 176 disposed around the circumference of the dermal cap 160 adjacent to the rounded periphery surface 172. The pre-cut holes 176 are configured to facilitate using soft tissue anchors or sutures to retain the position and configuration of the dermal cap 160 on the bone head being treated.

Similarly to the dermal cap 100, described in connection with FIG. 1, the dermal cap 160 can be adapted for treating the metatarsal head 112, shown in FIG. 3, or the metacarpal head 120, shown in FIG. 4, to restore joint function. As such, the dermal cap 160 may be implemented in various sizes to accommodate the anatomy of the target bone joints. In some embodiments, the dermal cap 160 has a diameter that ranges between about 10 mm and about 24 mm. Further, the dermal cap 160 may be non-perforated as shown in FIG. 2. In some embodiments, however, the dermal cap 160 may be perforated to aid bone tissue with fusing to the dermal cap 160. Further, the dermal cap 160 may comprise any synthetic or natural homogenous material suitable for implantation into bone, including human or animal allograft, and the like.

While the dermal cap and methods have been described in terms of particular variations and illustrative figures, those of ordinary skill in the art will recognize that the dermal cap is not limited to the variations or figures described. In addition, where methods and steps described above indicate certain events occurring in certain order, those of ordinary skill in the art will recognize that the ordering of certain steps may be modified and that such modifications are in accordance with the variations of the dermal cap. Additionally, certain of the steps may be performed concurrently in a parallel process, when possible, as well as performed sequentially as described above. To the extent there are variations of the dermal cap, which are within the spirit of the disclosure or equivalent to the dermal cap found in the claims, it is the intent that this patent will cover those variations as well. Therefore, the present disclosure is to be understood as not limited by the specific embodiments described herein, but only by scope of the appended claims.

What is claimed is:

1. An apparatus for a dermal cap for treating osteochondral defects of metatarsal bones of the feet or metacarpal bones of the hands, the dermal cap comprising:
an outer surface having a convex shape;
an inner surface having a concave shape; and
a rounded peripheral surface joining the inner surface and the outer surface.

2. The dermal cap of claim 1, wherein the inner surface is configured to be placed into contact with a metatarsal head or a metacarpal head such that the outer surface provides a bone head surface resembling the original articular cartilage.

3. The dermal cap of claim 2, wherein the outer surface is configured to effectively restore the articular cartilage and thus serves to relieve pain and facilitate better joint function, as well as potentially delay or prevent an onset of arthritis.

4. The dermal cap of claim 1, wherein the dermal cap is configured to be formed by stretching it over a rounded mandrel that is shaped similarly to the size and anatomy of a bone head to be treated.

5. The dermal cap of claim 4, wherein the dermal cap is configured to be freeze-dried on the rounded mandrel to set the desired anatomy of the dermal cap.

6. The dermal cap of claim 1, further including pre-cut holes disposed around a circumference of the dermal cap adjacent to the rounded periphery surface.

7. The dermal cap of claim 6, wherein the pre-cut holes are configured to facilitate using soft tissue anchors or sutures to retain the position and configuration of the dermal cap on the bone head being treated.

8. The dermal cap of claim 1, further including a tab and a pre-cut hole that are configured to facilitate using soft tissue anchors or sutures to mount the dermal cap onto the bone head being treated.

9. The dermal cap of claim 1, wherein the dermal cap is configured to be implemented in various sizes to accommodate the anatomy of the specific bone joints to be treated.

10. The dermal cap of claim 1, wherein the dermal cap has a diameter that ranges between about 10 mm and about 24 mm.

11. The dermal cap of claim 1, wherein the dermal cap is non-perforated.

12. The dermal cap of claim 1, wherein the dermal cap is perforated to aid bone tissue with fusing to the material comprising the dermal cap.

13. The dermal cap of claim 12, wherein the material comprising the dermal cap comprise any synthetic or natural homogenous material suitable for implantation into bone.

14. The dermal cap of claim 13, wherein the material comprises human or animal allograft.

15. A method for a dermal cap for treating osteochondral defects of metatarsal bones of the feet or metacarpal bones of the hands, comprising:
forming an outer surface having a convex shape;
forming an inner surface having a concave shape; and
joining the inner surface and the outer surface by way of a rounded peripheral surface.

16. The method of claim 15, wherein forming the inner surface includes forming the inner surface to be placed into contact with a metatarsal head or a metacarpal head such that the outer surface provides a bone head surface resembling the original articular cartilage.

17. The method of claim 16, wherein forming the outer surface includes configuring the outer surface to restore the articular cartilage and thus to relieve pain and facilitate better joint function, as well as potentially delay or prevent an onset of arthritis.

18. The method of claim 15, further including disposing pre-cut holes around a circumference of the dermal cap adjacent to the rounded periphery surface.

19. The method of claim 18, wherein disposing the pre-cut holes includes configuring the pre-cut holes to facilitate using soft tissue anchors or sutures to retain the position and configuration of the dermal cap on the bone head being treated.

20. The method of claim 15, further including configuring a tab and a pre-cut hole to facilitate using soft tissue anchors or sutures to mount the dermal cap onto the bone head being treated.
